(19) United States
(12) Patent Application Publication
(10) Pub. No.: US 2025/0059145 A1
(43) Pub. Date: Feb. 20, 2025

(54) SOLID FORMS OF 1-{3-[3-(4-CHLOROPHENYL) PROPOXY] PROPYL} PIPERIDINE HYDROCHLORIDE AND PROCESS FOR THE PREPARATION THEREOF

(71) Applicant: BIOPHORE INDIA PHARMACEUTICALS PVT. LTD., Hyderabad (IN)

(72) Inventors: Manik Reddy PULLAGURLA, Hyderabad (IN); Kiran Kumar KOTHAKONDA, Hyderabad (IN); Bhaskar Reddy PITTA, Hyderabad (IN); Rajesham BOGE, Hyderabad (IN); Jagadeesh Babu RANGISETTY, Hyderabad (IN)

(21) Appl. No.: 18/723,502

(22) PCT Filed: Dec. 29, 2022
(86) PCT No.: PCT/IB2022/062865

§ 371 Date: Jun. 24, 2024

(30) Foreign Application Priority Data
Dec. 29, 2021 (IN) 202141061496

(51) Int. Cl. C07D 295/088; A61K 9/14; A61K 31/4453

(52) U.S. Cl. CPC C07D 295/088; A61K 9/146; A61K 31/4453

(57) ABSTRACT

Solid forms of Pitolisant or its salt are provided. Also provided are solid dispersions of Pitolisant or salt thereof in amorphous form and at least one suitable pharmaceutically acceptable excipient. The process for preparing solid dispersion of Pitolisant or salt thereof is also provided.

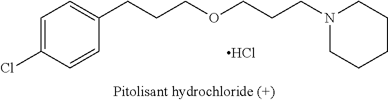 [SMILES: Clc1ccc(CCCOCCCN2CCCCC2)cc1 ; Cl]

### CROSS-REFERENCE TO RELATED APPLICATIONS

This application is a national stage of PCT Application No. PCT/IB2022/062865, having a filing date of Dec. 29, 2022, which claims priority to IN 202141061496, having a filing date of Dec. 29, 2021, the entire contents both of which are hereby incorporated by reference.

### FIELD OF TECHNOLOGY

The following relates to a solid dispersion of Pitolisant or its salt (1) in amorphous form and at least one pharmaceutically acceptable excipient. Further the following also relates to a process for preparing solid dispersion comprising Pitolisant or a salt thereof in amorphous form and at least one pharmaceutically acceptable excipient.

### BACKGROUND

Pitolisant hydrochloride (1) is indicated for the treatment of excessive daytime sleepiness (EDS) in adult patients with narcolepsy. Pitolisant hydrochloride is chemically described as 1-{3-[3-(4-chlorophenyl) propoxy]propyl}piperidine hydrochloride. It is white crystalline powder. It was approved by USFDA under the brand name WAKIX.

The synthesis of Pitolisant hydrochloride (1) has been reported in few patents the contents of which are hereby incorporated as reference in their entirety.

U.S. Pat. No. 8,207,197 patent discloses a process for the preparation of Pitolisant hydrochloride, its crystalline form and a pharmaceutical composition comprising the same. Synthesis of Pitolisant hydrochloride (1) is as shown in scheme 1, below.

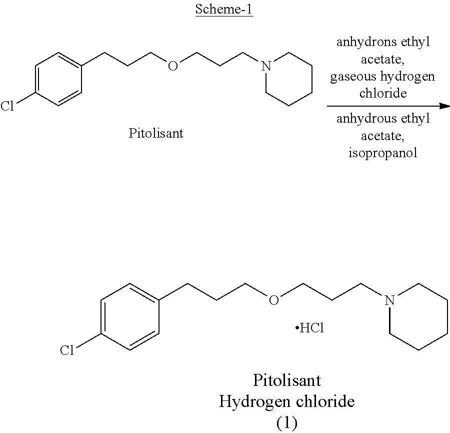 [SMILES: Clc1ccc(CCCOCCCN2CCCCC2)cc1 ; Clc1ccc(CCCOCCCN2CCCCC2)cc1 ; Cl]

Thus, there is need of preparing novel solid forms of Pitolisant hydrochloride (1) which show high solubility, stability and commercially viable. Hence, the present inventors, hereby disclose solid dispersions of Pitolisant or salt (1) in amorphous form with at least one pharmaceutically acceptable excipient.

### SUMMARY

An aspect relates to novel solid forms of Pitolisant or its salt (1).

In another aspect, embodiments of the present invention provide solid forms comprising of solid dispersions of Pitolisant or its salt (1) with at least one pharmaceutically acceptable excipient.

In another aspect, embodiments of the present invention provide a process for preparing solid dispersions of Pitolisant or its salt (1) in amorphous form and at least one pharmaceutically acceptable excipient.

In another aspect, embodiments of the present invention provide a process for the preparation of amorphous form of Pitolisant or its salt (1).

In another aspect, the solid form obtained by any of the described methods has purity more than 99.0% (w/w), for example, more than 99.5% (w/w) by HPLC.

Accordingly, in one aspect embodiments of the present invention provide solid forms of Pitolisant or its salt (1).

In another aspect, embodiments of the present invention provide solid forms comprising of solid dispersions of Pitolisant or its salt (1) and at least one pharmaceutically acceptable excipient.

In another aspect, embodiments of the present invention provide a process for preparing solid dispersions of Pitolisant or its salt (1) and at least one pharmaceutically acceptable excipient, comprising:
a) suspending Pitolisant or its salt (1) and at least one pharmaceutically acceptable excipient in a suitable solvent or mixture of solvents;
b) stirring the reaction mixture at a suitable temperature;
c) filtering the reaction mixture at a suitable temperature;
d) removing the solvent by suitable method; and
e) isolating solid dispersion (s) of Pitolisant or its salt (1) with at least one pharmaceutically acceptable excipient.

In another aspect, the solid dispersions of Pitolisant or salt (1) with at least one pharmaceutically acceptable excipient is characterized by powder X-ray powder diffraction.

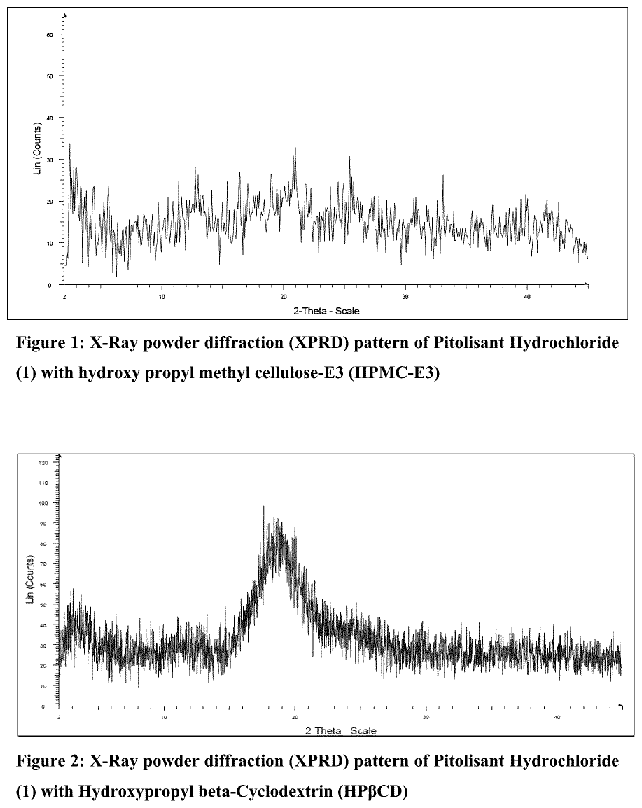

In another aspect, the solid dispersion of Pitolisant hydrochloride (1) with at least one pharmaceutically acceptable excipient, for example, hydroxy propyl methyl cellulose-E3 (HPMC-E3), Hydroxypropyl beta-Cyclodextrin (HPPCD) is characterized by X-ray powder diffraction pattern as shown in FIG. 1 and FIG. 2.

Another aspect of embodiments of the invention is to provide amorphous form of Pitolisant or its salt (1), comprising the following steps:
1. adding Pitolisant or its salt (1) to a suitable solvent or mixture of solvents;
2. heating the reaction mass to a suitable temperature;
3. cooling the reaction mass to a suitable temperature; and
4. isolating amorphous Pitolisant or its salt (1).

In another objective, solid forms obtained by any of the described methods have purity more than 99.0% (w/w), for example, more than 99.5% (w/w) by HPLC.

### BRIEF DESCRIPTION

Some of the embodiments will be described in detail, with references to the following Figures, wherein like designations denote like members, wherein:

FIG. 1 shows an X-Ray powder diffraction (XPRD) pattern of solid dispersion of Pitolisant hydrochloride (1) with hydroxy propyl methyl cellulose-E3 (HPMC-E3) prepared by example 1; and

FIG. 2 shows an X-Ray powder diffraction (XPRD) pattern of solid dispersion of Pitolisant hydrochloride (1) with Hydroxypropyl beta-Cyclodextrin (HPDCD) prepared by example 3.

### DETAILED DESCRIPTION

Accordingly, embodiments of the present invention provide solid forms of Pitolisant or its salt (1).

In another embodiment, the present invention provide solid forms that may be comprising of solid dispersions of Pitolisant or its salt (1) and at least one suitable pharmaceutically acceptable excipient. Further, physical mixtures of solid dispersions of Pitolisant or its salt (1) with a suitable pharmaceutically acceptable excipient.

In some embodiments, the present invention provides a process for preparing solid dispersions of Pitolisant or salt (1) and at least one pharmaceutically acceptable excipient, comprising:
a) suspending Pitolisant or salt (1) and at least one pharmaceutically acceptable excipient in a suitable solvent or mixture of solvents;
b) stirring the reaction mixture at a suitable temperature;
c) filtering the reaction mixture at a suitable temperature;
d) removing the excess solvent; and
e) isolating solid dispersion of Pitolisant or its salt (1) and at least one pharmaceutically acceptable excipient using a suitable technique.

In some embodiments, the present invention provides a process for the preparation of solid dispersions of Pitolisant hydrochloride (1) by suspending Pitolisant hydrochloride (1) and a suitable pharmaceutically acceptable excipient in a suitable solvent and stirring to a suitable temperature of 20-40° C., for example, 25-30° C. The reaction mixture may be filtered, and the solvent of the filtrate may be removed by a suitable technique. The solid dispersions of Pitolisant hydrochloride (1) and a suitable pharmaceutically acceptable excipient may be isolated using a suitable technique.

In another embodiment, the solid dispersions of Pitolisant or its salt (1) and at least one pharmaceutically acceptable excipient is characterized by PXRD.

In another embodiment, the solid dispersion of Pitolisant or salt (1) and at least one pharmaceutically acceptable excipient, for example, Hydroxypropyl beta-Cyclodextrin (HPβCD) and hydroxy propyl methyl cellulose-E3 (HPMC-E3), is characterized by X-ray powder diffraction pattern as shown in FIG. 1 and FIG. 2.

In another embodiment, the solid dispersion of Pitolisant or slat (1) and at least one pharmaceutically acceptable excipient obtained is amorphous or sometimes crystalline.

In another embodiment of the present invention a process to provide amorphous form of Pitolisant or its salt (1) comprises the following steps:
1. adding Pitolisant or its salt (1) to a suitable solvent or mixture of solvents;
2. heating the reaction mass to a suitable temperature;
3. cooling the reaction mass to a suitable temperature; and
4. isolating amorphous Pitolisant or its salt (1).

In some embodiments, the present invention provides a process for the preparation of amorphous form of Pitolisant hydrochloride (1), by suspending Pitolisant hydrochloride (1) in a suitable solvent and heating to a suitable temperature of 25-50° C., for example 40-45° C. The solvent may be removed and Pitolisant hydrochloride (1) is analyzed by PXRD, which shown amorphous form.

In some embodiments, Pitolisant or salt (1) as described above, wherein salt is selected from acid addition salt such as hydrochloric acid, hydrobromic acid, nitric, carbonic, monohydrogencarbonic, phosphoric, monohydrogenphosphoric, dihydrogen phosphoric, sulfuric, monohydrogensulfuric, hydriodic, or phosphorous acids and the like, as well as the salts derived from organic acids such as acetic acid, propionic acid, isobutyric acid, malonic acid, benzoic acid, succinic acid, suberic acid, fumaric acid, mandelic acid, phthalic acid, benzenesulfonic acid, p-tolylsulfonic acid, citric acid, tartaric acid, methansulfonic acid, and the like.

In some embodiments, the suitable pharmaceutically acceptable excipients used may be selected from a group comprising of lactose, maltose, sucrose, sorbitol, mannitol, polysorbate, maltodextrin, saccharose, cellulose, methyl cellulose, ethyl cellulose, microcrystalline cellulose (MCC), polyethylene glycol (PEG), polyethylene glycol-4000 (PEG-4000), polyvinylpyrrolidone (PVP), polyvinylpyrrolidone K-30, (PVP K-30), Copovidone, povidone, magnesium stearate, polyvinyl acetate hydroxyethyl cellulose (HEC), hydroxy propyl methyl cellulose (HPMC), hydroxypropyl cellulose (HPC), hydroxy propyl methyl cellulose acetate succinate (HPMC-AS), hydroxy propyl methyl cellulose-E3, (HPMC-E3), soluplus, Neusilin, Eudragit, Eudragit-EPO, Dicalcium phosphate (DCP), croscarmellose, sodium croscarmellose, α-cyclodextrin, β-Cyclodextrin, γ-cyclodextrin, hydroxypropyl beta cyclodextrin (HPBCD), sulfobutylether-β-cyclodextrin (SBCED) or the like. For example, hydroxypropyl beta cyclodextrin (HPBCD), Eudragit, polyvinylpyrrolidone K-30 (PVP K-30), hydroxy propyl methyl cellulose-E3 (HPMC-E3), Eudragit-EPO, hydroxy propyl methyl cellulose-E3 (HPMC-E3), hydroxypropyl beta cyclodextrin (HPBCD), Neusilin, Dicalcium phosphate (DCP), may be used in embodiments of the present invention.

In another embodiment, the ratio of Pitolisant hydrochloride (1) and the pharmaceutically acceptable excipients used in the preparation of physical mixture may range from 1:1 to 1:5, for example, 1:2 was used in embodiments.

The suitable solvents used herein may be selected from but not limited to a group comprising of water, methanol, ethanol, n-propanol, iso-propanol, n-butanol, iso-butanol, 2-butanol, tert-butanol, n-hexane, n-heptane, cyclohexane, pet ether, benzene, toluene, dimethyl ether, diethyl ether, diisopropyl ether, methyl tert-butyl ether, 1,2-dimethoxyethane, tetrahydrofuran, 1,4-dioxane, methyl acetate, ethyl acetate, n-propyl acetate, isopropyl acetate, n-butyl acetate, isobutyl acetate, tert-butyl acetate, dimethylformamide, dichloromethane, dichloroethane, chloroform, carbon tetrachloride, acetone, methyl ethyl ketone, acetonitrile, propionitrile, or mixtures thereof. For example, methanol and dichloromethane were used in embodiments of the present invention.

In another embodiment the solid forms obtained may be isolated by a suitable technique comprising of but not limited to filtration, precipitation, cooling, re-crystallization, concentrating the mass, distillation, flash evaporation, simple evaporation, fast solvent evaporation, rotational drying, spray drying, thin-film drying, agitated thin-film drying, freeze-drying or the like, for example, distillation was used in embodiments of the present invention.

The suitable techniques used for isolating the desired product may be selected from but not limited to filtration, solvent evaporation, lyophilization or freeze drying, spray drying, agitated thin film drying (ATFD), air tray drying (ATD), Vacuum tray dryer (VTDR), vacuum drying, cooling the solvent, adding anti-solvent to the reaction mixture and combination thereof. For example, Vacuum tray dryer (VTDR) was used in embodiments of the present invention. The drying can be carried out at a suitable temperature ranging from 40° C. to about 70° C., for example, from 60° C. to about 65° C.

In another embodiment, the solid dispersions of Pitolisant hydrochloride (1) obtained by any of the above method may have purity more than 99.0% (w/w), for example more than 99.5% (w/w).

In another embodiment, the solid dispersions of Pitolisant hydrochloride (1) obtained by any of the above method may have moisture content not more than 10% (w/w), for example, not more than 5% (w/w), or as a further example, not more than 3% (w/w).

### EXAMPLES

### Example-1: Preparation of solid dispersion of Pitolisant hydrochloride (1) with hydroxy propyl methyl cellulose-E3 (HPMC-E3) of formula (2)

250 mg of Pitolisant hydrochloride (1) and 500 mg of hydroxy propyl methyl cellulose-E3 (HPMC-E3) were suspended in 20 mL of water at 25-30° C. The suspension was stirred for 10-15 mins and transferred to a petri dish. The petri plate was kept in freeze dryer. The solid was further dried in vacuum tray dryer (VTD) below 45° C. to yield solid dispersion of Pitolisant hydrochloride (1). The resulting solid was analyzed by PXRD which showed amorphous form. Yield: 95% (w/w). PXRD: FIG. 1.

### Example-2: Preparation of Solid Dispersion of Pitolisant Hydrochloride (1) with Polyvinylpyrrolidone-K-30 (PVP-K-30) of Formula (3)

250 mg of Pitolisant hydrochloride (1) and 500 mg of polyvinylpyrrolidone-K-30 (PVP-K-30) were suspended in 20 mL of water at 25-30° C. The suspension was stirred for 10-15 minutes at room temperature and transferred to a petri dish. The petri plate was kept in freeze dryer. The solid was further dried in vacuum tray dryer (VTD) below 45° C. to yield solid dispersion of Pitolisant hydrochloride (1) in amorphous form. Yield: 96% (w/w).

### Example-3: Preparation of Solid Dispersion of Pitolisant Hydrochloride (1) with Hydroxypropyl Beta-Cyclodextrin (HPβCD) of Formula (4)

250 mg of Pitolisant hydrochloride (1) and 500 mg of Hydroxypropyl beta-Cyclodextrin (HPBCD) were suspended in 50 mL of water at 25-30° C. The suspension was stirred for 10-15 mins at room temperature and transferred to a petri dish. The petri plate was kept in freeze dryer for lyophilization. The solid was further dried in vacuum tray dryer (VTD) below 45° C. to yield solid dispersion of Pitolisant hydrochloride (1). The resulting solid was analyzed by PXRD which showed amorphous form. Yield: 95% (w/w). PXRD: FIG. 2.

### Example-4: Preparation of Solid Dispersion of Pitolisant Hydrochloride (1) with Polyethylene Glycol-4000 (PEG-4000) of Formula (5)

250 mg of Pitolisant hydrochloride (1) and 500 mg of Polyethylene glycol-4000 (PEG-4000) were added to 60 mL of water at 25-30° C. The suspension was stirred for 10-15 mins and transferred to a petri dish. The petri plate was kept in freeze dryer for lyophilization. The solid was further dried in vacuum tray dryer (VTD) below 45° C. to yield amorphous solid dispersion of Pitolisant hydrochloride (1). Yield: 95% (w/w).

### Example-5: Preparation of Solid Dispersion of Pitolisant Hydrochloride (1) with Lactose of Formula (6)

250 mg of Pitolisant hydrochloride (1) and 500 mg of Lactose were added in 20 mL of water at 25-30° C. The suspension was stirred for 10-15 mins at room temperature and transferred to a petri dish. The petri plate was kept in freeze dryer for lyophilization. The solid was further dried in vacuum tray dryer (VTD) below 45° C. to yield solid dispersion of Pitolisant hydrochloride (1). Yield: 950% (w/w).

### Example-6: Preparation of Solid Dispersion of Pitolisant Hydrochloride (1) with Maltose of Formula (7)

250 mg of Pitolisant hydrochloride (1) and 500 mg of Maltose were added to 60 mL of water at 25-30° C. The suspension was stirred for 10-15 mins and transferred to a petri dish. The petri plate was kept in freeze dryer for lyophilization. The solid was further dried in vacuum tray dryer (VTD) below 45° C. to yield amorphous solid dispersion of Pitolisant hydrochloride (1). Yield: 95% (w/w).

### Example-7: Preparation of Solid Dispersion of Pitolisant Hydrochloride (1) with Sorbitol of Formula (8)

250 mg of Pitolisant hydrochloride (1) and 500 mg of Sorbitol were added to 30 mL of water at 25-30° C. The suspension was stirred for 10-15 mins and transferred to a petri dish. The petri plate was kept in freeze dryer for lyophilization for 3 days. The solid was further dried in vacuum tray dryer (VTD) below 45° C. to yield amorphous solid dispersion of Pitolisant hydrochloride (1). Yield: 950% (w/w).

### Example-8: Preparation of Solid Dispersion of Pitolisant Hydrochloride (1) with Polysorbate of Formula (9)

250 mg of Pitolisant hydrochloride (1) and 500 mg of Polysorbate were added to 50 mL of water at 25-30° C. The suspension was stirred for 10-15 mins and transferred to a petri dish. The petri plate was kept in freeze dryer for lyophilization. The solid was further dried in vacuum tray dryer (VTD) below 45° C. to yield amorphous solid dispersion of Pitolisant hydrochloride (1). Yield: 96% (w/w).

### Example-9: Preparation of Solid Dispersion of Pitolisant Hydrochloride (1) with Maltodextrin of Formula (10)

250 mg of Pitolisant hydrochloride (1) and 500 mg of Maltodextrin were suspended in 20 mL of water at 25-30° C. The suspension was stirred for 10-15 mins and transferred to a petri dish. The petri plate was kept in freeze dryer for lyophilization for 3 days. The solid was further dried in vacuum tray dryer (VTD) below 45° C. to yield amorphous solid dispersion of Pitolisant hydrochloride (1). Yield: 96% (w/w).

### Example-10: Preparation of Solid Dispersion of Pitolisant Hydrochloride (1) with Copovidone of Formula (11)

250 mg of Pitolisant hydrochloride (1) and 500 mg of Copovidone were added in 40 mL of water at 25-30° C. The suspension was stirred for 10-15 mins and transferred to a petri dish. The petri plate was kept in freeze dryer for lyophilization for 3 days. The solid was further dried in vacuum tray dryer (VTD) below 45° C. to yield solid dispersion of Pitolisant hydrochloride (1). Yield: 95% (w/w).

### Example-11: Preparation of Solid Dispersion of Pitolisant Hydrochloride (1) with Hydroxypropyl Cellulose (HPC) of Formula (12)

250 mg of Pitolisant hydrochloride (1) and 500 mg of Hydroxypropyl cellulose (HPC) were added to 20 mL of dimethyl sulfoxide at 25-30° C. The suspension was stirred for 10-15 mins and transferred to a petri dish. The petri plate was kept in freeze dryer for lyophilization for 3 days. The solid was further dried in vacuum tray dryer (VTD) below 45° C. to yield amorphous solid dispersion of Pitolisant hydrochloride (1). Yield: 96% (w/w).

### Example-12: Preparation of Solid Dispersion of Pitolisant Hydrochloride (1) with Hydroxy Propyl Methyl Cellulose Acetate Succinate (HPMC-AS) of Formula (13)

250 mg of Pitolisant hydrochloride (1) and 500 mg of hydroxy propyl methyl cellulose acetate succinate (HPMC-AS) were added in 20 mL of dimethyl sulfoxide at 25-30° C. The suspension was stirred for 10-15 mins and transferred to a petri dish. The petri plate was kept in freeze dryer for lyophilization. The solid was further dried in vacuum tray dryer (VTD) below 45° C. to yield solid dispersion of Pitolisant hydrochloride (1). Yield: 96% (w/w).

### Example-13: Preparation of Solid Dispersion of Pitolisant Hydrochloride (1) with Eudragit of Formula (14)

250 mg of Pitolisant hydrochloride (1) and 500 mg of Eudragit were added to 20 mL of dimethyl sulfoxide at 25-30° C. The suspension was stirred for 10-15 mins and transferred to a petri dish. The petri plate was kept in freeze dryer for lyophilization for 3 days. The solid was further dried in vacuum tray dryer (VTD) below 45° C. to yield amorphous solid dispersion of Pitolisant hydrochloride (1). Yield: 96% (w/w).

### Example-14: Preparation of Solid Dispersion of Pitolisant Hydrochloride (1) with Soluplus of Formula (15)

250 mg of Pitolisant hydrochloride (1) and 500 mg of Soluplus were added to 20 mL of water at 25-30° C. The suspension was stirred for 10-15 mins and transferred to a petri dish. The petri plate was kept in freeze dryer for lyophilization for 3 days. The solid was further dried in vacuum tray dryer (VTD) below 45° C. to yield amorphous solid dispersion of Pitolisant hydrochloride (1). Yield: 94% (w/w).

### Example-15: Preparation of Solid Dispersion of Pitolisant Hydrochloride (1) with Mannitol of Formula (16)

250 mg of Pitolisant hydrochloride (1) and 500 mg of Mannitol was added to 25 mL of water at 25-30° C. The suspension was stirred for 10-15 mins and transferred to a petri dish. The petri plate was kept in freeze dryer for lyophilization for 3 days. The solid was further dried in vacuum tray dryer (VTD) below 45° C. to yield amorphous solid dispersion of Pitolisant hydrochloride (1). Yield: 94% (w/w).

### Example-16: Preparation of Solid Dispersion of Pitolisant Hydrochloride (1) with Sucrose of Formula (17)

250 mg of Pitolisant hydrochloride (1) and 500 mg of Sucrose were added in 15 mL of water at 25-30° C. The suspension was stirred for 10-15 mins and transferred to a petri dish. The petri plate was kept in freeze dryer for lyophilization for 3 days. The solid was further dried in vacuum tray dryer (VTD) below 45° C. to yield amorphous solid dispersion of Pitolisant hydrochloride (1). Yield: 94% (w/w).

### Example-17: Preparation of Solid Dispersion of Pitolisant Hydrochloride (1) with Polyvinylpyrrolidone K-30, (PVP K-30) of Formula (18)

5 g of Pitolisant hydrochloride (1) was added to 150 mL of methanol at 25-30° C. 10 g of polyvinylpyrrolidone-K-30 (PVP-K-30) was added to the reaction mass and stirred for 15-20 mins. The total reaction mixture was then transferred to a beaker and spray dried with the below settings:
Inlet Temperature: 50° C.
Outlet Temperature: 30° C.
Aspirator: 70%
Feed Rate: 20 ml/min
N₂ Pressure 2.0 kg/cm²

The obtained solid was unloaded and dried in Vacuum air tray dryer (VTD) to yield amorphous solid dispersion of Pitolisant hydrochloride (1) with polyvinylpyrrolidone-K-30 (PVP-K-30). Yield: 89% (w/w).

### Example-18: Preparation of Solid Dispersion of Pitolisant Hydrochloride (1) with Hydroxypropyl Beta Cyclodextrin (HPBCD) of Formula (19)

5 g of Pitolisant hydrochloride (1) was added to 150 mL of mixture of methanol, dichloromethane (1:1) at 25-30° C. 10 g of hydroxypropyl beta cyclodextrin (HPBCD) was added to the reaction mixture and stirred for 15-20 minutes. The reaction mass was then transferred to a beaker and spray dried with the below settings:
Inlet Temperature: 50° C.
Outlet Temperature: 30° C.
Aspirator: 70%
Feed Rate: 20 ml/min
N₂ Pressure 2.0 kg/cm²

The obtained solid was unloaded and dried in Vacuum air tray dryer (VTD) to yield amorphous solid dispersion of Pitolisant hydrochloride (1) hydroxypropyl beta cyclodextrin (HPBCD). Yield: 65% (w/w).

### Example-19: Preparation of Solid Dispersion of Pitolisant Hydrochloride (1) with Hydroxypropyl Beta Cyclodextrin (HPBCD) of Formula (20)

200 g of Pitolisant free base (2) and 600 ml of water were taken at room temperature. Cooled the reaction mass to 0-5° C. 36 ml of conc. Hydrochloric acid was added slowly at 0-5° C. and raised the temperature to 25-30° C. 300 g of Hydroxypropyl β-cyclodextrin (HPβCD) was added at the same temperature and stirred for 20-30 minutes. Passed the reaction mass through 0.2-micron filter at room temperature. Filtrate was loaded into the freeze dryer and dry the solid for 44-48 hours at 20-25° C. to get a title compound. Yield: 92%; Purity: >99.9%

Although the invention has been illustrated and described in greater detail with reference to the exemplary embodiments, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

1-8. (canceled)

9. A solid dispersion of Pitolisant or its salt and at least one pharmaceutically acceptable excipient.

10. A process for preparing a solid dispersion comprising Pitolisant or its salt and at least one pharmaceutically acceptable excipient with purity greater than 99.5% (w/w) by High-performance liquid chromatography, comprising:
a) suspending Pitolisant or its salt and a suitable pharmaceutically acceptable excipient(s) in a suitable solvent or mixture of solvents;
b) stirring the reaction mixture at a suitable temperature;
c) filtering the reaction mixture at a suitable temperature;
d) removing the excess solvent; and
e) isolating solid dispersion (s) of Pitolisant or its salt with a suitable pharmaceutically acceptable excipient using a suitable technique.

11. The process according to claim 10, wherein the solid dispersion is an amorphous solid dispersion comprising Pitolisant or salt and at least one pharmaceutically acceptable excipient, the method comprising:
i. suspending Pitolisant or salt and Hydroxypropyl beta cyclodextrin (HPBCD) in a suitable solvent or mixture of solvents;
ii. stirring the reaction mixture at a suitable temperature;
iii. filtering the reaction mixture at a suitable temperature;
iv. removing the excess solvent by freeze dryer; and
v. isolating the amorphous solid dispersion of Pitolisant or its saltusing a suitable technique.
wherein the resulting solid was analysed by PXRD.

12. The solid dispersion as claimed in claim 9, wherein the at least one pharmaceutically acceptable excipient is selected from the group comprising of lactose, maltose, sucrose, sorbitol, mannitol, polysorbate, maltodextrin, saccharose, cellulose, methyl cellulose, ethyl cellulose, microcrystalline cellulose, polyethylene glycol, polyethylene glycol-4000, polyvinylpyrrolidone, polyvinylpyrrolidone K-30,, Copovidone, povidone, magnesium stearate, polyvinyl acetate hydroxyethyl cellulose, hydroxy propyl methyl cellulose, hydroxypropyl cellulose, hydroxy propyl methyl cellulose acetate succinate, hydroxy propyl methyl cellulose-E3,, soluplus, Neusilin, Eudragit, Eudragit-EPO, Dicalcium phosphate, croscarmellose, sodium croscarmellose, α-cyclodextrin, β-Cyclodextrin, γ-cyclodextrin, hydroxypropyl beta cyclodextrin, sulfobutylether-β-cyclodextrin or the like.

13. The process as claimed in claim 10, wherein the at least one pharmaceutically acceptable excipient is selected from the group comprising of lactose, maltose, sucrose, sorbitol, mannitol, polysorbate, maltodextrin, saccharose, cellulose, methyl cellulose, ethyl cellulose, microcrystalline cellulose, polyethylene glycol, polyethylene glycol-4000, polyvinylpyrrolidone, polyvinylpyrrolidone K-30,, Copovidone, povidone, magnesium stearate, polyvinyl acetate hydroxyethyl cellulose, hydroxy propyl methyl cellulose, hydroxypropyl cellulose, hydroxy propyl methyl cellulose acetate succinate, hydroxy propyl methyl cellulose-E3,, soluplus, Neusilin, Eudragit, Eudragit-EPO, Dicalcium phosphate, croscarmellose, sodium croscarmellose, α-cyclodextrin, β-Cyclodextrin, γ-cyclodextrin, hydroxypropyl beta cyclodextrin, sulfobutylether-β-cyclodextrin or the like.

14. The solid dispersion as claimed in claim 9, wherein the solid dispersion comprises Pitolisant or its salt and the at least one pharmaceutically acceptable excipient ratio in a weight range of from 1:1 to 1:5.

15. A process for preparing amorphous form of Pitolisant or its salt, having purity greater than 99.5% (w/w) by High-performance liquid chromatography, the process comprising:
1. adding Pitolisant or its salt to a suitable solvent or mixture of solvents;
2. heating the reaction mass to a suitable temperature;
3. cooling the reaction mass to a suitable temperature; and
4. isolating amorphous Pitolisant or its salt.

16. The process as claimed in claim 10, wherein the solvent is selected from water, methanol, ethanol, n-propanol, iso-propanol, n-butanol, iso-butanol, 2-butanol, tert-butanol, n-hexane, n-heptane, cyclohexane, pet ether, benzene, toluene, dimethyl ether, diethyl ether, diisopropyl ether, methyl tert-butyl ether, 1,2-dimethoxyethane, tetrahydrofuran, 1,4-dioxane, methyl acetate, ethyl acetate, n-propyl acetate, isopropyl acetate, n-butyl acetate, isobutyl acetate, tert-butyl acetate, dimethylformamide, dichloromethane, dichloroethane, chloroform, carbon tetrachloride, acetone, methyl ethyl ketone, acetonitrile, propionitrile, and its mixtures thereof.

17. The process as claimed in claim 15, wherein the solvent is selected from water, methanol, ethanol, n-propanol, iso-propanol, n-butanol, iso-butanol, 2-butanol, tert-butanol, n-hexane, n-heptane, cyclohexane, pet ether, benzene, toluene, dimethyl ether, diethyl ether, diisopropyl ether, methyl tert-butyl ether, 1,2-dimethoxyethane, tetrahydrofuran, 1,4-dioxane, methyl acetate, ethyl acetate, n-propyl acetate, isopropyl acetate, n-butyl acetate, isobutyl acetate, tert-butyl acetate, dimethylformamide, dichloromethane, dichloroethane, chloroform, carbon tetrachloride, acetone, methyl ethyl ketone, acetonitrile, propionitrile, and its mixtures thereof.

18. The process as claimed in claim 10, wherein the isolation technique used is selected from filtration, precipitation, cooling, re-crystallization, concentrating the mass, distillation, flash evaporation, simple evaporation, fast solvent evaporation, rotational drying, spray drying, thin-film drying, agitated thin-film drying, freeze-drying.

19. The process as claimed in claim 15, wherein the isolation technique used is selected from filtration, precipitation, cooling, re-crystallization, concentrating the mass, distillation, flash evaporation, simple evaporation, fast solvent evaporation, rotational drying, spray drying, thin-film drying, agitated thin-film drying, freeze-drying.
